(19) United States
(12) Patent Application Publication
(10) Pub. No.: US 2021/0375414 A1
(43) Pub. Date: Dec. 2, 2021

(54) APPARATUS AND METHODS FOR GENERATION OF A MEDICAL SUMMARY

(71) Applicant: DIAGNOSTICS ROBOTICS LTD., Tel Aviv - Jaffa (IL)

(72) Inventors: Yonatan Amir, Jerusalem (IL); Hanan Rokach, Ashkelon (IL); Daniel Glozman, Kfar Yona (IL); Guy Elad, Kiryat Tivon (IL)

(21) Appl. No.: 17/285,275

(22) PCT Filed: Sep. 27, 2019
(86) PCT No.: PCT/IB2019/058214

§ 371 Date: Apr. 14, 2021

Related U.S. Application Data
(60) Provisional application No. 62/745,480, filed on Oct. 15, 2018.

(51) Int. Cl. G16H 15/00; G16H 10/20; G16H 10/60

(52) U.S. Cl. CPC G16H 15/00; G16H 10/60; G16H 10/20

(57) ABSTRACT

Apparatus and methods are described for generating a medical summary of a patient. Using a patient-interface device (24), a plurality of questions are outputted to the patient, and respective answers are received to the plurality of questions, from the patient. Using at least one computer processor (26), for each of the answers received from the patient, a corresponding question sub-template is generated, and the question sub-templates are embedded into respective pre-defined locations within a medical summary template. A healthcare-professional interface device (30) is driven to display a medical summary of the patient in prose form, using the question sub-templates embedded within the medical summary template. Other applications are also described.

### CROSS-REFERENCE TO RELATED APPLICATION

The present application claims priority from U.S. Provisional patent application 62/745,480 to Amir, filed Oct. 15, 2018, entitled “Apparatus and methods for generation of a medical summary,” which is incorporated herein by reference.

### FIELD

The invention relates to methods and apparatus for use in emergency rooms and in other clinical settings, and particularly apparatus and methods that are used to increase the diagnostic accuracy and patient throughput during emergency room or clinical procedures.

### BACKGROUND

The execution of emergency room medicine is usually described as being made up of five elements: identification, triage, anamnesis (i.e., obtaining a preliminary medical history relating to the patient), diagnosis, and prognosis.

Emergency room medicine is characterized by there being a need for speedy handling and diagnosis of patients. Speedy diagnoses are especially important for medical conditions relating to cerebrovascular incidents, strokes, and cardiac events. The requirement of providing speedy diagnoses is compounded by the need to commonly operate in high load situations, especially in situations of mass casualty events, as well as extreme weather conditions, when elderly and weak patients are highly prone to illness. Despite the conditions of urgency and the high load in a typical emergency room setting, it is necessary to provide diagnoses with a relatively high degree of accuracy, since the accuracy of the diagnosis can have a bearing on decisions regarding priority of treatment, which could have repercussions on the survival rate both of the patient being examined and of other patients. Incorrect diagnosis is a common cause of death in the medical field, and many such events occur as a result of a delay in treatment or incorrect diagnosis in the setting of the emergency room.

### SUMMARY

In accordance with some applications of the invention, methods and apparatus and are provided for at least partially automating the process of receiving a patient's details, details of the patient's present medical state, and/or the patient's medical history, and generating a medical summary that is readable by a healthcare professional. Typically, a patient-questionnaire station outputs questions to the patient, and receives answers to the questions from the patient. At least partially based upon the answers that the patient provides, a computer processor generates a medical summary of the patient, and drives a healthcare-professional interface device to display the medical summary. For example, the medical summary may be displayed upon a monitor that is used by a doctor and/or a nurse, and/or a print-out of the medical summary to be read by a doctor and/or a nurse may be generated. Alternatively or additionally, the medical summary is automatically entered into the hospital's electronic record system and/or the patient's electronic medical records. For some applications, a list of patients in the emergency room is presented upon a dashboard, and healthcare professionals are able to see a given patient's automatically-generated medical summary by selecting that patient from the list.

Typically, the medical summary is generated in prose form. For some applications, the computer processor is configured to generate the medical summary in a language that is different from the language in which the questions were output to the patient, and/or in a language that is different from the language in which the patient inputted their responses to the questions. In this manner, if, for example, a patient is not a native speaker of the language that is used by the doctor, the patient is able to receive questions in a language that he/she understands and to respond in that language, while the doctor is able to receive the medical summary in the language that he/she best understands.

There is therefore provided, in accordance with some applications of the invention, a method for generating a medical summary of a patient, the method including:

using a patient-interface device, outputting a plurality of questions to the patient, and receiving respective answers from the patient to the plurality of questions; and

using at least one computer processor:
for each of the answers received from the patient, generating a corresponding question sub-template;
embedding the question sub-templates into respective pre-defined locations within a medical summary template; and
driving a healthcare-professional interface device to display a medical summary of the patient in prose form, using the question sub-templates embedded within the medical summary template.

There is further provided, in accordance with some applications of the invention, a computer software product, for generating a medical summary of a patient, and for use with a patient-interface device and a healthcare-professional interface device, the computer software product including a non-transitory computer-readable medium in which program instructions are stored, which instructions, when read by a computer cause the computer to perform the steps of:

driving the patient-interface device to output a plurality of questions to the patient and to receive respective answers from the patient to the plurality of questions;

for each of the answers received from the patient, generating a corresponding question sub-template;

embedding the question sub-templates into respective pre-defined locations within a medical summary template; and

driving the healthcare-professional interface device to display a medical summary of the patient in prose form, using the question sub-templates embedded within the medical summary template.

There is further provided, in accordance with some applications of the invention, apparatus for generating a medical summary of a patient, the apparatus including:

a healthcare-professional interface device;

a patient-interface device configured to output a plurality of questions to the patient and to receive respective answers from the patient to the plurality of questions; and

at least one computer processor configured:
for each of the answers received from the patient, to generate a corresponding question sub-template;
to embed the question sub-templates into respective pre-defined locations within a medical summary template; and
to drive the healthcare-professional interface device to display a medical summary of the patient in prose form, using the question sub-templates embedded within the medical summary template.

In some applications, the computer processor is configured:

to drive the patient-interface device to output the plurality of questions to the patient, and to receive respective answers from the patient to the plurality of questions, in a first language, and

to drive the healthcare-professional interface device to display the medical summary of the patient in prose form, in a second language that is different from the first language.

In some applications, the computer processor is configured to embed the question sub-templates into respective pre-defined locations within the medical summary template by:

based upon at least one symptom that the patient indicates that they are suffering from, selecting a symptom representative template; and

embedding the question sub-templates into respective pre-defined locations within the symptom representative template.

In some applications, the computer processor is further configured, in response to receiving a given answer from the patient to at least one of the questions, to generate an alert on the healthcare-professional interface device.

In some applications, the computer processor is further configured to automatically add the medical summary into an electronic medical record of the patient.

In some applications, the computer processor is further configured, at least partially in response to receiving one or more of the answers from the patient, to generate a first output selected from the group consisting of: an output indicating that the patient should be hospitalized, an output indicating a triage score for the patient, an output recommending that one or more tests be performed on the patient, and an output indicating a differential diagnosis of the patient.

In some applications, the computer processor is further configured to generate a second output indicating which data relating to the patient led to generating the first output.

In some applications, the computer processor is further configured to update the first output in real time, in response to receiving additional data relating to the patient.

In some applications, the computer processor is configured to generate the first output in response to performing machine-learning analysis with respect to the one or more answers.

In some applications, the computer processor is configured to automatically receive additional data relating to the patient, and to drive the healthcare-professional interface device to display the medical summary of the patient in prose form by incorporating at least some of the additional data into the medical summary.

In some applications, the computer processor is configured to automatically receive additional data relating to the patient by automatically receiving medical records of the patient.

In some applications, the computer processor is configured to automatically receive additional data relating to the patient by automatically receiving a result of a test performed upon the patient.

In some applications, the computer processor is configured to embed the question sub-templates into respective pre-defined locations within the medical summary template by, in response to the patient not providing an answer to one of the questions, not including text corresponding to the question sub-template within the medical summary template.

In some applications, the computer processor is configured, in a designated section within the medical summary, to indicate that the patient did not provide an answer to the question.

There is further provided, in accordance with some applications of the invention, a method for generating a medical summary of a patient, the method including:

using a patient-interface device, outputting a plurality of questions to the patient in a first language, and receiving respective answers from the patient to the plurality of questions in the first language;

using at least one computer processor, generating a medical summary of the patient in a second language that is different from the first language, the medical summary being at least partially based upon the answers that were received from the patient; and using the computer processor, driving a healthcare-professional interface device to display the medical summary of the patient in the second language.

There is further provided, in accordance with some applications of the invention, a computer software product, for generating a medical summary of a patient, and for use with a patient-interface device and a healthcare-professional interface device, the computer software product including a non-transitory computer-readable medium in which program instructions are stored, which instructions, when read by a computer cause the computer to perform the steps of:

using the patient-interface device, outputting a plurality of questions to the patient in a first language, and receiving respective answers from the patient to the plurality of questions in the first language;

generating a medical summary of the patient in a second language that is different from the first language, the medical summary being at least partially based upon the answers that were received from the patient; and

driving the healthcare-professional interface device to display the medical summary of the patient in the second language.

There is further provided, in accordance with some applications of the invention, apparatus for generating a medical summary of a patient, the apparatus including:

a healthcare-professional interface device;

a patient-interface device; and

at least one computer processor configured:
using the patient-interface device, to output a plurality of questions to the patient in a first language, and to receive respective answers from the patient to the plurality of questions in the first language;
to generate a medical summary of the patient in a second language that is different from the first language, the medical summary being at least partially based upon the answers that were received from the patient; and
to drive the healthcare-professional interface device to display the medical summary of the patient in the second language.

In some applications, the computer processor is configured to generate the medical summary in the second language by:

for each of the answers received from the patient, generating a corresponding question sub-template; and

embedding the question sub-templates into respective pre-defined locations within a medical summary template.

In some applications, the computer processor is configured to generate the medical summary in the second language by using known translations of each of the question sub-templates and the medical summary template.

In some applications, the computer processor is configured to embed the question sub-templates into respective pre-defined locations within the medical summary template by:

based upon at least one symptom that the patient indicates that they are suffering from, selecting a symptom representative template; and

embedding the question sub-templates into respective pre-defined locations within the symptom representative template.

In some applications, the computer processor is configured to embed the question sub-templates into respective pre-defined locations within the medical summary template by, in response to the patient not providing an answer to one of the questions, not including text corresponding to the question sub-template within the medical summary template.

In some applications, the computer processor is configured to generate the medical summary by, in a designated section within the medical summary, indicating that the patient did not provide an answer to the question.

The invention will be more fully understood from the following detailed description of applications thereof, taken together with the drawings, in which:

### BRIEF DESCRIPTION OF THE DRAWINGS

FIG. 1 is a schematic illustration of a patient interacting with a patient-questionnaire station in an emergency room setting, in accordance with some applications of the invention;

FIG. 2 is a flowchart indicating a typical flow of a patient questionnaire, in accordance with some applications of the invention; and

FIG. 3 is a flowchart showing steps of a method that is performed, in accordance with some applications of the invention.

### DETAILED DESCRIPTION

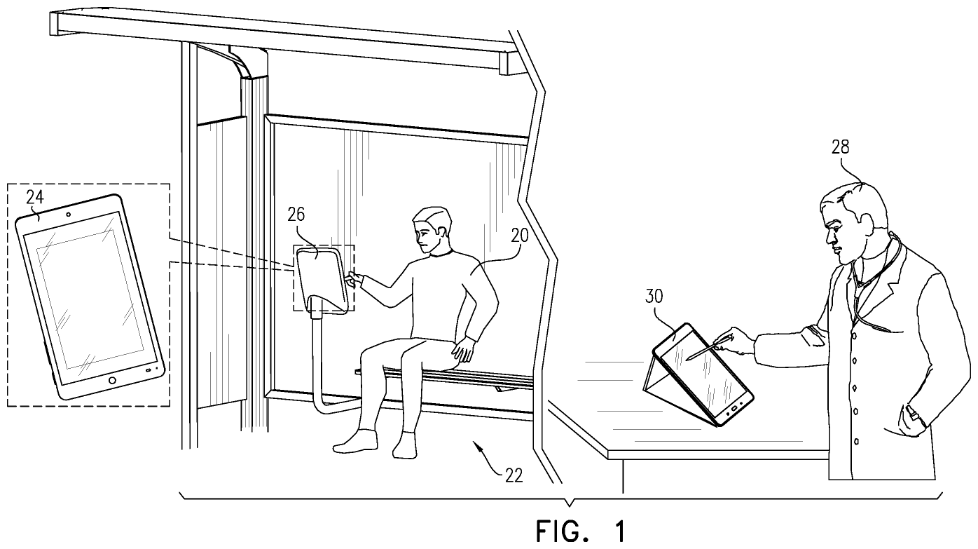

Reference is now made to FIG. 1, which is a schematic illustration of a patient 20 interacting with a patient-questionnaire station 22, in accordance with some applications of the invention. Typically, the patient-questionnaire station is disposed within a triage section of an emergency room. The patient-questionnaire station typically includes a patient-interface device 24, which outputs questions to the patient, and receives answers to the questions from the patient. At least partially based upon the answers that the patient provides, a computer processor generates a medical summary of the patient and drives a healthcare-professional interface device 30 to display the medical summary. For example, the medical summary may be displayed upon a monitor that is used by a doctor 28 and/or a nurse, and/or a print-out of the medical summary to be read by a doctor and/or a nurse may be generated. Alternatively or additionally, the medical summary is automatically entered into the hospital's electronic record system and/or the patient's electronic medical records. For some applications, a list of patients in the emergency room is presented upon a dashboard, and the healthcare professionals are able to see a given patient's automatically-generated medical summary by selecting that patient from the list.

Typically, the medical summary is generated in prose form. For some applications, the computer processor is configured to generate the medical summary in a language that is different from the language in which the questions were output to the patient, and/or in a language that is different from the language in which the patient inputted their responses to the questions. In this manner, if, for example, a patient is not a native speaker of the language that is used by the doctor, the patient is able to receive questions in a language that he/she understands and to respond in that language, while the doctor is able to receive the medical summary in the language that he/she best understands.

Typically, the patient-questionnaire station includes a computer processor 26, which communicates with a memory (not shown), and with patient-interface device 24. The computer processor typically drives an output unit of the patient-interface device 24 to output questions to the patient, and receives the patient's inputs from an input unit of the patient-interface device. For some applications, the input unit of the patient-interface device includes a keyboard, a mouse, a joystick, a touchscreen device (such as a smartphone or a tablet computer), a touchpad, a trackball, a voice-command interface, and/or other types of input devices that are known in the art. For some applications, the output portion of the patient-interface device includes a display, and/or a different type of visual, text, graphics, tactile, audio, and/or video output device, e.g., speakers, headphones, a smartphone, or a tablet computer. Typically, the healthcare-professional interface device 30 includes generally similar hardware components to those described with reference to the patient-interface device.

As described hereinabove, a computer processor generates a medical summary of the patient and drives healthcare-professional interface device 30 to display the medical summary. For some applications, computer processor 26 of the patient-questionnaire station is configured to perform the aforementioned steps. Alternatively or additionally, one or more additional computer processors (not shown) that are in communication with computer processor 26 are configured to perform these steps. For example, additional computer processors that are connected to the hospital server may be configured to perform these steps, and/or computer processors that are outside the hospital and disposed within a remote server may be configured to perform these steps. For some applications, any one of the aforementioned computer processors is configured to analyze the patient's responses using machine-learning techniques and to generate a diagnosis of the patient in response thereto (e.g., using techniques as described in WO 18/220565 to Amir, which is incorporated herein by reference). For some applications, any one of the aforementioned computer processors is configured to generate an alert, in response to a given response or set of responses from a patient. For example, in response to a patient indicating that he/she is over a certain age and is suffering from severe chest pain, the computer processor may generate an alert on a healthcare professional interface device indicating that the patient is in urgent need of an assessment by a doctor. In accordance with respective applications, the computer processor generates outputs on an interface device associated with a given healthcare professional, and/or a given set of healthcare professionals, e.g., as described hereinabove. For some applications, the computer processor generates an output on a computer-readable medium (e.g., a non-transitory computer-readable medium), such as a disk, or a portable USB drive, and/or generates an output on a printer.

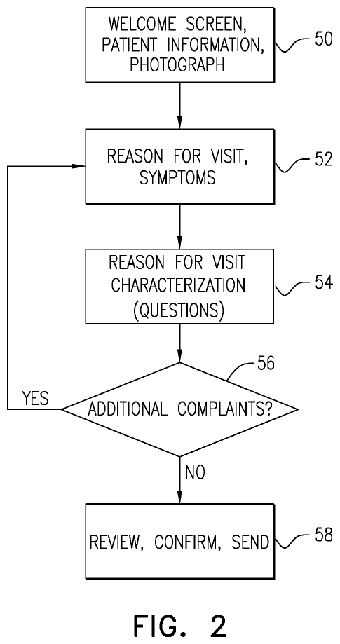

Reference is now made to FIG. 2, which is a flowchart indicating a typical flow of a patient questionnaire, in accordance with some applications of the invention. Typically, the questionnaire starts with a welcome screen, in which the patient is asked to enter his/her details, and optionally a photograph of the patient is taken (box 50). Subsequently, the patient is asked to enter the reason for the visit to the emergency room, and/or symptoms (box 52). As described hereinbelow, for some applications, a medical summary template is selected by selecting a symptom template representation based upon the main symptoms that the patient presents. For some applications, the symptom template representation is selected based upon the inputs provided by the patient during the phase of the questionnaire corresponding to box 52. Typically, the questionnaire then proceeds with more questions in which the patient's condition is further characterized (box 54). Subsequently the questionnaire proceeds by asking the patient to enter any additional complaints, and/or details thereof (box 56). Once the patient has completed the questionnaire, the patient typically is provided with an opportunity to review the responses that he/she provided, and to confirm the responses, before sending the responses into the system for analysis (box 58).

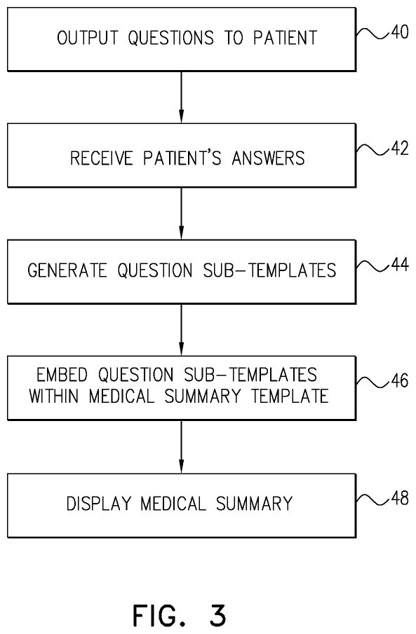

Reference is now made to FIG. 3, which is a flowchart showing steps of a method that is performed, in accordance with some applications of the invention. For some applications, in a first step 40, a plurality of questions are outputted to the patient, via the patient-interface device. In step 42, respective answers to the plurality of questions are received from the patient, via the patient-interface device. Typically, the questions belong to different categories, which include multiple choice questions (i.e., questions to which the patient may select a plurality of answers out of multiple options), single-choice questions (i.e., questions to which the patient must select only one answer out of multiple options), yes/no/don't-know questions (i.e., questions to which the patient must select either “yes,” “no,” or “don't know”), scale questions (i.e., questions to which the patient has to rate something, such as pain level, on a scale). An example of a set of questions and corresponding answers from a patient who is suffering from chest pain is shown in Table 1 below.

TABLE 1
Example of questions and corresponding answers

| Question | | |
|---|---|---|
| number | Question | Answer |
| 1 | Enter ID | XXXXXX |
| 2 | Enter Age | 57 |
| 3 | Select Sex | Female |
| 4 | Select Complaint/Symptom | Chest Pain |
| 5 | Have you recently had a chest injury (as a | No |
| | result of falling, car accident, etc.)? | |
| 6 | How long ago did the pain begin? | Approximately |
| | | 24 hours ago |
| 7 | How does the pain change with time? | Constant pain |
| 8 | Please describe your pain | Stabbing |
| 9 | Please rate your chest pain on a scale from 1 | 9 |
| | to 10 (10 - most painful) | |
| 10 | Please specify the location of the pain | Center of the |
| | | chest |
| 11 | Is the pain focused or spread? | Focused |
| 12 | Does the chest pain spread or radiate to any | Left arm, Left |
| | of these areas? | shoulder |
| 13 | Does any of the following make the pain | Exercise |
| | worse? | |
| 14 | Does any of the following relieve the pain? | Rest |
| 15 | Do you have any additional symptoms? | Headache, |
| | | Weakness |
| 16 | Do you suffer, or have you suffered in the | Coronary artery |
| | past, from the following cardiovascular | disease, High |
| | conditions? | blood pressure |
| 17 | Do you suffer, or suffered in the past, from | Rheumatoid |
| | the following conditions? | Arthritis |
| 18 | Do you smoke? | No |
| 19 | Do you drink alcohol? | No |
| 20 | Do you use recreational drugs? | No |
| 21 | To what medication, if any, do you have a | Penicillin |
| | sensitivity? | |
| 22 | What medication, if any, do you regularly | Aspirin, |
| | take? | Tritace |
| 23 | What surgery, if any, have you had | None |
| | performed in the past year? | |

In step 44, for each of the answers received by the patient, a corresponding question sub-template is generated, and in step 46, the question sub-templates are embedded into respective pre-defined locations within a medical summary template. Typically, the structure of the medical summary template depends on the main symptoms that the patient is suffering from. Further typically, based upon the main symptoms that the patient is suffering from, a “symptom template representation” is selected, the symptom template representation being a template that contains sections relating to information that is pertinent to such symptoms. The question sub-templates are then embedded within the selected symptom template representation, as described in further detail hereinbelow.

Steps 44 and 46 may be understood with reference to an example shown below of a symptom template representation and a corresponding medical summary that is the output of step 46:

### Example of Symptom Template Representation for a Patient Presenting with Chest Pain

[{The patient is a <answer_2> years old} {<answer_3>} {presenting with a chief complaint of <answer_4>}.]
[The pain *{started <answer_6>} {is <answer_11>} {is located to the <answer_10>}*] [The patient describes the pain as *,{<answer_7>} {<answer_8>} {at a scale of <answer_9>/10} {radiating to the <answer_12>}*] [The pain *,{is worsened by <answer_13>} {is relieved by <answer_14>}*] [{<answer_5>}.] [{The patient also describes <answer 15> and <answer_15>}]
[The patient *,{has known sensitivity to <answer_21>}, {takes <answer_22> <answer_22> regularly}*] [{Medical history includes <answer_16> <answer_16> and <answer_16>}] [{The patient also has a history of a <answer_17>}] [The patient *, {<answer_18>} {<answer_19>} {<answer_20>} {<answer_23>}*]

As shown, the symptom template representation typically includes sentence templates (denoted by [square brackets], in the example shown above), and question templates (denoted by {curly brackets}, in the example shown above), and question sub-templates (denoted by <angled brackets>, in the example shown above). The asterisk followed by a comma in the example shown above denote joining of question templates that allow for multiple answers to be entered, whereby the answers are then punctuated correctly with commas, the word “and” before the final entry, etc. A final asterisk denoted the end of such a sentence structure.

### Example of Medical Summary (which Corresponds to the Answers Shown in Table 1 Above)

The patient is a 57 years old female presenting with a chief complaint of chest pain.
The pain started approximately 24 hours ago and is focused and located to the center of the chest. The patient describes the pain as constant, stabbing, and is a scale of 9/10, radiating to the left arm and left shoulder. The pain is worsened by exercise and is relieved by rest. There was no recent chest injury. The patient also describes headache and weakness.
The patient has known sensitivity to penicillin, and takes Aspirin and Tritace regularly. Medical history includes high blood pressure, coronary artery disease, and asthma. The patient also has a history of a rheumatoid arthritis. The patient doesn't smoke and has no history of alcohol and no history of drug abuse, and has had no previous surgeries in the last year.

As indicated in the above examples, typically the questions in the symptom template representation and the corresponding medical summary are divided into three categories: demographics, history of present illness, and past medical history. For some applications, the three categories are presented in three separate paragraphs, as shown above. It is noted that the example shown above is for when the patient presents with one main symptom. In case in which there are multiple main symptoms, the medical summary typically includes a corresponding number of paragraphs relating to the history of the present illness, with one paragraph corresponding to each of the main symptoms.

As described hereinabove, in step 44, for each of the answers received by the patient, a corresponding question sub-template is generated. Typically, the question category imposes properties that appear in the corresponding question sub-template. For example, for the multiple-choice questions, the question sub-template may (a) contain wording corresponding to the multiple choice question and allow the patient's answer(s) to become embedded within that wording, but (b) also allow for that wording to be modified if the patient answers “None of the above.” By way of example, the multiple choice question may be “Does the chest pain spread or radiate to any of these areas?” and the corresponding multiple-choice answers may be (i) Left arm, (ii) Left shoulder, (iii) Right Arm, (iv) Right shoulder, and (v) None of the above. If the patient selects any one of the first four answers, then the patient's answer is embedded in text of the question sub-template, which states “radiating to <answer>.” However, if the patient answers “None of the above,” then the text of this question sub-template will indicate that the pain does not radiate. For some applications, if a patient doesn't answer a given question, indicated that they didn't understand the question, and/or indicated that they didn't know the answer, the corresponding question sub-template is deleted from the medical summary. Typically, in such cases, an indication of this having happened is provided to the healthcare professional in a separate section of the medical summary.

As described hereinabove, in step 46, the question sub-templates are embedded into respective pre-defined locations within a medical summary template. Typically, the structure of the medical summary template depends on the main symptoms that the patient is suffering from. For example, as described hereinabove, a symptom template representation may be selected based upon the main symptoms that the patient presents, and the question sub-templates may then be arranged within the selected symptom template representation. Typically, the questions that the patient is asked are selected at least partially based upon the answers that patient provided to previous questions, e.g., as described in WO 18/220565 to Amir, which is incorporated herein by reference. In the above example, the questions that were asked and the medical summary that was generated are pertinent to a patient that is suffering from chest pain. However, had the patient initially presented with a suspected broken leg, then the medical summary template would be structured accordingly, and, for example, may include a section relating to how the injury came about, whether the patient is able to walk on the leg, whether there is bruising on the leg, etc.

Referring again to FIG. 3, in step 48, based on the patient's answers to the questions, the computer processor drives a healthcare-professional interface device to display a medical summary of the patient in prose form, using the question sub-templates embedded within the medical summary template. Typically, the medical summary is based upon a symptom template representation, which is selected based upon the main symptoms that the patient presents, as described hereinabove. For some applications, in order to generate the medical summary the computer processor accesses data relating to the patient from additional sources. For example, the computer processor may connect to electronic medical records of the patient, based upon having received identifying information regarding the patient. Such data relating to the patient may include historical information regarding the patient, such as previous hospital visits, medication the patient has taken, symptoms the patient has suffered from, conditions that the patient has, etc. For some applications, the computer processor accesses the results of tests that are performed on the patient, e.g., tests that are performed in real time during the patient's current visit to the hospital. Such tests may include blood tests, ECG, medical imaging, and/or any tests described in WO 18/220565 to Amir, filed May 31, 2018, which is incorporated herein by reference.

As described hereinabove, for some applications, the computer processor is configured to generate the medical summary in a language that is different from the language in which the questions were output to the patient, and/or in a language that is different from the language in which the patient inputted their responses to the questions. It is noted that in accordance with the above description, the medical summary is typically built from predefined portions of text. For example, the question sub-templates and the sentence templates of the medical summary template are typically predefined. The computer processor is typically preprogrammed such that the translations all of the options for each of the question sub-templates are known, and the translations of the sentence templates are known. Thus, in order to perform the translation, the computer processor translates each of the components of the medical summary using the known translations of these components. Typically, the result of performing the translation in this manner is that the translated medical summary is a more accurate and readable medical summary, than, for example, if the entire medical summary in the first language was simply to be machine translated to the second language.

For some applications, in addition to generating a patient medical summary, based upon the inputs from the patient (and/or additional data, e.g., as described hereinabove), the computer processor is configured to (a) recommend whether the patient shall be hospitalized, (b) provide a triage score, (c) suggest medical tests, (d) provide a differential diagnosis, and/or (e) provide a patient score. For some applications, in addition to providing one or more of the aforementioned outputs, the computer processor is configured to generate an indication of which data led the computer processor to generate such an output (e.g., which data led the computer processor to recommend that the patient should be hospitalized, and/or to provide a differential diagnosis). For some applications, the computer processor performs such techniques using machine-learning techniques. Typically, the computer processor updates the outputs in real time, in response to receiving additional data (e.g., additional information from the patient, additional test results, etc.) For some applications, such techniques are performed in conjunction with techniques described in WO 18/220565 to Amir, which is incorporated herein by reference.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 26. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 26) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the invention may be written in any combination of one or more programming languages, including an object-oriented programming language such as Java, Smalltalk, C++ or the like and conventional procedural programming languages, such as the C programming language or similar programming languages.

It will be understood that blocks of the flowcharts shown in the figures and combinations of blocks in the flowcharts, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 26) or other programmable data processing apparatus, create means for implementing the functions/acts specified in the flowcharts and/or algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowcharts and/or algorithms described in the present application.

Computer processor 26 is typically a hardware device programmed with computer program instructions to produce a special purpose computer. For example, when programmed to perform the algorithms described with reference to the figures, computer processor 26 typically acts as a special purpose patient-analysis computer processor. Typically, the operations described herein that are performed by computer processor 26 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the invention is not limited to what has been particularly shown and described hereinabove. Rather, the scope of the invention includes both combinations and subcombinations of the various features described hereinabove, as well as variations and modifications thereof that are not in the prior art, which would occur to persons skilled in the art upon reading the foregoing description.

1. A method for generating a medical summary of a patient, the method comprising:
using a patient-interface device, outputting a plurality of questions to the patient, and receiving respective answers from the patient to the plurality of questions; and
using at least one computer processor:
for each of the answers received from the patient, generating a corresponding question sub-template;
embedding the question sub-templates into respective pre-defined locations within a medical summary template; and
driving a healthcare-professional interface device to display a medical summary of the patient in prose form, using the question sub-templates embedded within the medical summary template.

2. The method according to claim 1, wherein:
outputting the plurality of questions to the patient, and receiving respective answers from the patient to the plurality of questions comprises outputting the plurality of questions to the patient and receiving respective answers from the patient to the plurality of questions, in a first language, and
driving the healthcare-professional interface device to display the medical summary of the patient in prose form comprises driving the healthcare-professional interface device to display the medical summary of the patient in prose form, in a second language that is different from the first language.

3. The method according to claim 1, wherein embedding the question sub-templates into respective pre-defined locations within a medical summary template comprises:
based upon at least one symptom that the patient indicates that they are suffering from, selecting a symptom representative template; and
embedding the question sub-templates into respective pre-defined locations within the symptom representative template.

4. The method according to claim 1, further comprising, in response to receiving a given answer from the patient to at least one of the questions, generating an alert on the healthcare-professional interface device.

5. The method according to claim 1, further comprising automatically adding the medical summary into an electronic medical record of the patient.

6. The method according to claim 1, further comprising, at least partially in response to receiving one or more of the answers from the patient, generating a first output selected from the group consisting of: an output indicating that the patient should be hospitalized, an output indicating a triage score for the patient, an output recommending that one or more tests be performed on the patient, and an output indicating a differential diagnosis of the patient.

7. The method according to claim 6, further comprising generating a second output indicating which data relating to the patient led to generating the first output.

8. The method according to claim 6, further comprising updating the first output in real time, in response to receiving additional data relating to the patient.

9. The method according to claim 6, wherein generating the first output at least partially in response to receiving one or more of the answers from the patient comprises generating the first output in response to performing machine-learning analysis with respect to the one or more answers.

10. The method according to claim 1, further comprising automatically receiving additional data relating to the patient, wherein driving the healthcare-professional interface device to display the medical summary of the patient in prose form comprises incorporating at least some of the additional data into the medical summary.

11. The method according to claim 10, wherein automatically receiving the additional data relating to the patient comprises automatically receiving medical records of the patient.

12. The method according to claim 10, wherein automatically receiving the additional data relating to the patient comprises automatically receiving a result of a test performed upon the patient.

13. The method according to claim 1, wherein embedding the question sub-templates into respective pre-defined locations within a medical summary template comprises, in response to the patient not providing an answer to one of the questions, not including text corresponding to the question sub-template within the medical summary template.

14. The method according to claim 13, further comprising, in a designated section within the medical summary, indicating that the patient did not provide an answer to the question.

15. A computer software product, for generating a medical summary of a patient, and for use with a patient-interface device and a healthcare-professional interface device, the computer software product comprising a non-transitory computer-readable medium in which program instructions are stored, which instructions, when read by a computer cause the computer to perform the steps of:
driving the patient-interface device to output a plurality of questions to the patient and to receive respective answers from the patient to the plurality of questions;
for each of the answers received from the patient, generating a corresponding question sub-template;
embedding the question sub-templates into respective pre-defined locations within a medical summary template; and
driving the healthcare-professional interface device to display a medical summary of the patient in prose form, using the question sub-templates embedded within the medical summary template.

16. An apparatus for generating a medical summary of a patient, the apparatus comprising:
a healthcare-professional interface device;
a patient-interface device configured to output a plurality of questions to the patient and to receive respective answers from the patient to the plurality of questions; and
at least one computer processor configured:
for each of the answers received from the patient, to generate a corresponding question sub-template;
to embed the question sub-templates into respective pre-defined locations within a medical summary template; and
to drive the healthcare-professional interface device to display a medical summary of the patient in prose form, using the question sub-templates embedded within the medical summary template.

17. The apparatus according to claim 16, wherein the computer processor is configured:
to drive the patient-interface device to output the plurality of questions to the patient, and to receive respective answers from the patient to the plurality of questions, in a first language, and
to drive the healthcare-professional interface device to display the medical summary of the patient in prose form, in a second language that is different from the first language.

18. The apparatus according to claim 16, wherein the computer processor is configured to embed the question sub-templates into respective pre-defined locations within the medical summary template by:
based upon at least one symptom that the patient indicates that they are suffering from, selecting a symptom representative template; and
embedding the question sub-templates into respective pre-defined locations within the symptom representative template.

19. The apparatus according to claim 16, wherein the computer processor is further configured, in response to receiving a given answer from the patient to at least one of the questions, to generate an alert on the healthcare-professional interface device.

20. The apparatus according to claim 16, wherein the computer processor is further configured to automatically add the medical summary into an electronic medical record of the patient.

21. The apparatus according to claim 16, wherein the computer processor is further configured, at least partially in response to receiving one or more of the answers from the patient, to generate a first output selected from the group consisting of: an output indicating that the patient should be hospitalized, an output indicating a triage score for the patient, an output recommending that one or more tests be performed on the patient, and an output indicating a differential diagnosis of the patient.

22. The apparatus according to claim 21, wherein the computer processor is further configured to generate a second output indicating which data relating to the patient led to generating the first output.

23. The apparatus according to claim 21, wherein the computer processor is further configured to update the first output in real time, in response to receiving additional data relating to the patient.

24. The apparatus according to claim 21, wherein the computer processor is configured to generate the first output in response to performing machine-learning analysis with respect to the one or more answers.

25. The apparatus according to claim 16, wherein the computer processor is configured to automatically receive additional data relating to the patient, and to drive the healthcare-professional interface device to display the medical summary of the patient in prose form by incorporating at least some of the additional data into the medical summary.

26. The apparatus according to claim 25, wherein the computer processor is configured to automatically receive additional data relating to the patient by automatically receiving medical records of the patient.

27. The apparatus according to claim 25, wherein the computer processor is configured to automatically receive additional data relating to the patient by automatically receiving a result of a test performed upon the patient.

28. The apparatus according to claim 16, wherein the computer processor is configured to embed the question sub-templates into respective pre-defined locations within the medical summary template by, in response to the patient not providing an answer to one of the questions, not including text corresponding to the question sub-template within the medical summary template.

29. The apparatus according to claim 28, wherein the computer processor is configured, in a designated section within the medical summary, to indicate that the patient did not provide an answer to the question.

30-42. (canceled)
